(19) United States
(12) Patent Application Publication
(10) Pub. No.: US 2018/0312798 A1
(43) Pub. Date: Nov. 1, 2018

(54) METHOD FOR FED-BATCH FERMENTATION OF CHLORELLAE FED BY SEQUENTIAL, AUTOMATED PROVISIONS OF GLUCOSE

(71) Applicant: Corbion Biotech, Inc., South San Francisco, CA (US)

(72) Inventors: Laurent Segueilha, Marquette Lez Lille (FR); Marie Le Ruyet, Lille (FR); Sylvain Delaroche, Longuenesse (FR)

(21) Appl. No.: 15/963,945

(22) Filed: Apr. 26, 2018

Related U.S. Application Data
(63) Continuation of application No. 14/911,078, filed on Feb. 9, 2016.

(30) Foreign Application Priority Data
Jul. 26, 2013 (FR) 1357387

(51) Int. Cl. C12N 1/12; C12P 7/64

(52) U.S. Cl. CPC C12N 1/12; C12P 7/64

(57) ABSTRACT

The invention related to a method for producing microalgae of the chlorella genus by fed-batch fermentation, characterized in that the provision of a carbon source is carried out sequentially and automatically in response to a drop in oxygen consumption by the microalga, in particular when the dissolved oxygen pressue in the fermentation medium (pO₂) exceeds a predefined threshold value.

### CROSS-REFERENCE TO RELATED APPLICATION

This application is a continuation of U.S. patent application Ser. No. 14/911,078, entitled “METHOD FOR FED-BATCH FERMENTATION OF CHLORELLAE FED BY SEQUENTIAL, AUTOMATED PROVISIONS OF GLUCOSE”, filed Feb. 9, 2016, which is the U.S. National Stage Application of International Patent No. PCT/FR2014/051943, filed Jul. 25, 2014, which claims the benefit of priority to French National Application No. 1357387, filed Jul. 26, 2013.

The present invention relates to a novel process for the production of microalgae of the genus Chlorella by fed-batch fermentation.

### Presentation of the State of the Art

Historically requiring “only water and sunlight” to grow, algae have for a long time been considered to be a source of food.

There exist several species of algae which can be used in food, the majority being “macroalgae”, such as kelp, sea lettuce (Ulva lactuca) and red algae of Porphyra (cultivated in Japan) or dulse (Palmaria palmata) type.

However, in addition to these macroalgae, there are also other sources of algae represented by the “microalgae”, that is to say photosynthetic or nonphotosynthetic unicellular microscopic algae, of or not of marine origin, cultured for their applications in biofuels or food.

For example, spirulina (Arthrospira platensis) is cultured in open lagoons (under phototropic conditions) for use as food supplement or incorporated in small amounts into confectionary or drinks (generally less than 0.5% weight/weight).

Other lipid-rich microalgae, including certain species belonging to the Chlorella genus, are also very popular in Asian countries as food supplements.

Several species of microalgae are capable of changing from photoautotrophic growth (by virtue of light, which supplies the energy for converting CO₂ into carbon-based chains) to heterotrophic growth (without light) using glucose or other carbon-based substrates which can be used for the metabolism of carbon and energy.

Three processes for the production of microalgae are currently used industrially:
in heterotrophic reactors (entirely closed);
in open-air ponds;
in glass tubes.

Chlorellae with variable properties and compositions are produced from these methods of culturing. The compositions will be different according to whether or not they are produced in light and whether or not they are produced in the open air.

The production and the use of the flour of microalgae of Chlorella type are, for example, described in the documents WO 2010/120923 and WO 2010/045368.

The oil fraction of the microalgal flour, which can be composed essentially of monounsaturated oils, can offer nutritional and health advantages in comparison with the saturated, hydrogenated and polyunsaturated oils often found in conventional foodstuffs.

When it is desired to industrially manufacture microalgal flour powders from their biomass, major difficulties remain, not only from the technological viewpoint but also from the viewpoint of the sensory profile of the compositions produced.

This is because, while algal powders, for example manufactured with algae photosynthetically cultured in open-air ponds or by photobioreactors, are available commercially, they have a dark green color (associated with chlorophyll) and a strong unpleasant taste.

In point of fact, it is thus generally accepted that the formation and the growth of chloroplasts are suppressed under heterotrophic culturing conditions and in darkness.

Under these heterotrophic conditions, the microalgae thus do not use the photosynthesis reaction but grow by consuming the sugars of the culture medium.

The advantages of this system of production are:
a greatly increased productivity by volume, multiplied by 100 with respect to an open system and by 10 with respect to the bioreactors,
very high concentrations of dry matter (hundreds of grams per liter),
low production costs,
products obtained of very high quality,
a confined medium and thus no contamination,
no constraint with regard to locality,
very easy to operate industrially,
a technology which has been completely mastered on an industrial scale with regard to yeasts and bacteria for several decades, by different industries, such as the chemical industry and the food-processing industry,
absence of chlorophyll and thus more neutral taste.

Two forms of heterotrophic culturing are conventionally described in the literature (for example by H. Iwamoto in Richmond, A. (ed.), 2004, Handbook of Microalgal Culture. Blackwell, Oxford, 255-263).

H. Iwamoto writes that, when the heterotrophic culturing stage is controlled in batch mode (supplying all the glucose all at once at the start of fermentation), the initial phase of exponential growth is followed by a stage of maturing, so as to obtain cells rich in advantageous compounds.

The biomass increases during the initial phase, with consumption of glucose, and then stops at zero glucose.

The second “maturing” phase is subsequently taken advantage of in order to promote the production of other advantageous molecules (pigments, lipids, and the like).

Culturing in fed-batch mode (gradual feeding with glucose) is generally carried out under “glucose-limiting” conditions.

The principle at the heart of the method, as described by H. Iwamoto, involves supplying glucose in response to its consumption by the growing Chlorellae.

The concentration of the glucose in the culture medium is analyzed continuously and automatically and is maintained at 1.5%.

Feeding with glucose is halted when the desired cell density is reached. Culturing is then maintained in this state for approximately 10 hours in order to promote cell maturation.

The application of this mode of fed-batch culturing under glucose-limiting conditions is illustrated by H. Iwamoto with a strain of Chlorella regularis.

Culturing is carried out for a total duration of 40 h, the first 30 hours being devoted to the growth of the microalgae and to feeding them with glucose.

The following 10 hours are devoted to the maturing, without supplying glucose.

The biomass is then collected and concentrated by centrifuging, washing, thermal inactivation (which makes it possible to inhibit chlorophyllase) at 130° C. for 3 seconds and dried by atomization, in order to obtain a very fine powder.

However, this way of proceeding is employed in particular to reactivate the production of chlorophyll and of carotenoids during the maturing stage.

It is not suitable for the production of lipid-rich Chlorella biomass without fault detrimentally affecting the organoleptic qualities (off notes) and in particular without production of chlorophyll.

Furthermore, the automatic devices for assaying the residual glucose are not sufficiently reliable and do not give a rapid enough response (>1 minute) to allow precise regulation of the fermentation.

An unsatisfied need thus remains to have available a method for effective production by fed-batch fermentation which is freed from the constraints of managing the residual glucose.

### SUMMARY OF THE INVENTION

The applicant company has found that it is possible to meet this need by providing a process for the production of microalgae of the Chlorella genus by fed-batch fermentation in which the additions of carbon-based source are sequential and automated under entirely specific conditions, that is to say by subjecting the supplying with the carbon-based source by sequential additions to the control of the value of the dissolved oxygen pressure (pO₂) of the fermentation medium.

The present invention thus relates to a process for the production of microalgae of the Chlorella genus by fed-batch fermentation, characterized in that the supplying with carbon-based source is carried out sequentially and automatically in response to a fall in the consumption of oxygen by the microalgae.

The microalga can be chosen from the group consisting of Chlorella protothecoides, Chlorella sorokiniana and Chlorella vulgaris. Preferably, the microalga is Chlorella protothecoides.

The carbon-based source can be any carbon source suitable for culturing by fermentation of the micro-algae. It can in particular be chosen from the group consisting of glucose, acetate and ethanol, and a mixture of these. Preferably, the carbon-based source is glucose.

The fall in the oxygen consumption of the microalga can be detected by measuring the dissolved oxygen pressure in the medium. As an increase in this pressure reflects a fall in the consumption, supplying with carbon-based source can be triggered when the dissolved oxygen pressure in the fermentation medium (pO₂) exceeds a threshold value.

This threshold value can be a pO₂ value from 1 to 100%, preferably from 1 to 80%, more preferably from 1 to 20%, more preferably still from 10 to 20%, greater than the pO₂ in the fermentation medium when the concentration of carbon-based source in the fermentation medium is not limiting.

Preferably, the pO₂ value established when the concentration of carbon-based source in the fermentation medium is not limiting is from 20 to 40% and more particularly preferably approximately 30%.

Preferably, the period of time between the moment when the carbon-based source has been completely consumed and supplying with carbon-based source is less than 5 minutes, more particularly preferably less than 1 minute.

The residual source of carbon is preferably maintained permanently, or virtually permanently, at a value of greater than 0 and less than 20 g/l, preferably less than 10 g/l.

Preferably, supplying with carbon-based source is carried out by means of a pump, the maximum flow rate of which makes it possible to add from 10 to 20 g/l of carbon-based source to the fermentation medium in less than 10 minutes.

### DETAILED DESCRIPTION OF THE INVENTION

Contrary to the technical preconception which claims that, in the case of a fed-batch fermentation, the concentration of residual glucose has to be maintained at between 5 and 15 g/l by continuous addition of glucose regulated by the automatic measurement of the concentration of residual glucose in the medium, the applicant company takes advantage of the observation according to which, when the glucose in the fermentation medium has been entirely consumed, the pO₂ rapidly increases.

The applicant company thus automates the supplying with carbon-based source by programming the glucose feed pump in order for the latter to trigger the glucose pulse at each rise in pO₂ and not when the concentration of residual glucose is below 5 g/l.

In other words, the glucose feed pump is triggered as soon as the measured value of the pO₂ is greater than a threshold value established with respect to the pO₂ measured when the concentration of residual glucose is not limiting.

This is reflected by the fact that the detection of the rise in the pO₂ beyond a predetermined threshold triggers the startup, preferably at its maximum speed, of the glucose feed pump for a predetermined time (preferably of less than 10 minutes) in order to contribute an amount of glucose corresponding to a concentration of between 1 and 30 g/l, preferably of approximately 10 g/l, corrected for the initial volume of the fermenter.

By virtue of this process, it is the microalga which directly and automatically “manages” its glucose requirements.

In comparison with the fed-batch fermentation process with continuous feeding with carbon-based source, the process according to the invention exhibits several advantages:
it does not require any human intervention, given that it makes possible precise and automatic regulation of the fermentation, as is demonstrated in the experimental part;
supplying with carbon-based source is carried out according to the requirements of the microorganisms. Thus, the fermentation medium can under no circumstances accumulate an amount of glucose greater than that defined by the pulses or, on the other hand, end up for a long time at zero residual glucose. These two parameters are essential for ensuring the quality of the finished product, in particular the absence of off notes, that is to say of detrimentally affected organoleptic characteristics;
the residual glucose is much better controlled and the fermentation protocol is thus more robust and more reproducible;
the productivity is at a maximum, without under-feeding with glucose and without accumulation of glucose at concentrations which inhibit the metabolism of the microalgae.

As used here, the term “productivity” corresponds to the amount of biomass manufactured per liter and per hour of fed-batch fermentation.

The conversion yield Yx/s conventionally represents the ratio of the biomass formed to the glucose consumed. However, within the meaning of the invention, in order to compare the various protocols tested, in particular in the experimental part, and to evaluate the impact of the modifications on the yield, the applicant company has chosen to rationalize this parameter by determining the value of yield for the production of 45% of fatty acids produced (as dry w/w of biomass).

The present invention thus relates to a process for the production of microalgae of the Chlorella genus by fed-batch fermentation, characterized in that supplying with carbon-based source is carried out sequentially and automatically in response to a fall in the consumption of oxygen by the microalga.

The carbon-based source can be any carbon source suitable for culturing by fermentation of the micro-algae. It can in particular be chosen from the group consisting of glucose, acetate and ethanol, and a mixture of these. Preferably, the carbon-based source is glucose.

In fed-batch fermentation, after the start of batch fermentation, that is to say without supplying, a carbon-based source is added throughout the fermentation process until a defined amount of biomass is obtained.

In contrast to the fed-batch processes of the prior art in which supplying with carbon-based source is carried out by continuous feeding, in the process according to the invention, supplying with carbon-based source is carried out by sequential additions or pulses.

These sequential additions or pulses consist, for each, of the addition of a large amount of a concentrated solution of the carbon-based source, preferably a glucose syrup, in a relatively short time, preferably of less than 10 min and particularly preferably approximately 6 min, in order to achieve the desired concentration in the fermentation must. Preferably, the desired concentration in the fermentation must is between 1 and 30 g/l, more particularly between 1 and 20 g/l and very particularly preferably between 10 and 20 g/l. According to a specific embodiment, the desired concentration of carbon-based source in the fermentation must is approximately 10 g/l. As used here, the term “approximately” refers to a value +/−20%, 10%, 5% or 2%.

As will be exemplified below, as soon as the glucose in the medium has been completely consumed and as soon as, consequently, the pO₂ increases, feeding is carried out with a concentrated glucose solution, for example a 700 g/l glucose solution, for a period of time of less minutes, in order to achieve a concentration of glucose in the fermentation medium of 1 to 20 g/l. Supplying with glucose is subsequently halted for the duration of consumption of the residual glucose. When the latter is consumed, the pO₂ rises, which triggers further supplying with glucose, and so on.

As mentioned above, these additions do not require specific human intervention, in contrast to continuous addition, during which it is necessary to make sure that the glucose feed flow rate indeed corresponds to the metabolic requirement of the strain (cf. FIG. 1). This is because, once the glucose has been consumed, the consumption of oxygen of the fermentation medium by the microalga will be suddenly reduced. Thus, according to the PID (proportional integral derivative) adjustments of the stirring or of the air flow rate or of the dome back pressure or of the supply of O₂, a significant rise of a few % in the pO₂ will take place. In the process according to the invention, it is this rise which automatically triggers supplying with glucose.

Supplying with carbon-based source is carried out by means of a pump, the maximum flow rate of which preferably makes it possible to add 10 to 20 g/l of carbon-based source to the fermentation medium in less than 10 minutes.

According to a specific embodiment, supplying with carbon-based source is carried out as soon as the pO₂ exceeds a threshold value which is from 1 to 100%, from 1 to 80%, from 5 to 80%, from 10 to 80% or preferably from 15 to 70% greater than the pO₂ value established when the concentration of carbon-based source in the fermentation medium is not limiting. According to a specific embodiment, supplying with carbon-based source is carried out as soon as the pO₂ exceeds a threshold value which is from 1 to 20%, more preferably still from 10 to 20% and more particularly preferably approximately 15% greater than the pO₂ value established when the concentration of carbon-based source in the fermentation medium is not limiting. According to another specific embodiment, supplying with carbon-based source is carried out as soon as the pO₂ exceeds a threshold value which is from 50 to 80%, more preferably still from 60 to 70% and more particularly preferably approximately 65% greater than the pO₂ value established when the concentration of carbon-based source in the fermentation medium is not limiting.

By way of example, a nonlimiting glucose concentration can be a concentration of between 5 and 15 g/l. The value of the pO₂ when the concentration of carbon-based source in the fermentation medium is not limiting depends on several parameters, including the PID (proportional integral derivative) adjustments of the stirring or of the air flow rate or of the dome back pressure or of the supply of O₂. These parameters are preferably unchanging throughout the fermentation phase intended to increase the biomass. Thus, the variations in pO₂ faithfully reflect the variations in oxygen consumption of the microalga.

The value of the pO₂ when the concentration of carbon-based source in the fermentation medium is not limiting is usually from 20 to 40%, preferably approximately 30%.

According to a specific embodiment, the pO₂ value when the concentration of carbon-based source in the fermentation medium is not limiting is approximately 30%, preferably 30%, and the threshold value which triggers supplying with carbon-based source is between approximately 35% and approximately 55%, preferably between 35% and 55%. According to another specific embodiment, the pO₂ value when the concentration of carbon-based source in the fermentation medium is not limiting is approximately 30%, preferably 30%, and the threshold value which triggers supplying with carbon source is approximately 35%, preferably 35%.

Preferably, the period of time between the moment when the carbon source has been completely consumed and supplying with carbon-based source in the fermentation medium is less than 5 minutes, very particularly preferably less than 1 minute. As indicated above, the complete consumption of the carbon-based source results in a sudden fall in the oxygen consumption, which is detected by the measurement of the value of the pO₂ in the fermentation medium by means of a specific probe, an increase in the pO₂ reflecting a fall in the oxygen consumption and thus a shortage of carbon source in the medium.

This reaction time thus makes it possible to keep the residual source of carbon virtually unchangingly at a value of greater than 0 and less than 20 g/l, preferably greater than 0 and less than 10 g/l, without risk of limiting or inhibiting the metabolism by the glucose.

The microalga can be any Chlorella suitable for fed-batch fermentation. According to one embodiment, the microalga is chosen from the group consisting of Chlorella protothecoides, Chlorella sorokiniana and Chlorella vulgaris. Preferably, the microalga is Chlorella protothecoides. According to a specific embodiment, the microalga is Chlorella protothecoides UTEX 250 (The Culture Collection of Algae at the University of Texas at Austin, USA).

A better understanding of the invention will be obtained using the examples which follow, which are meant to be illustrative and nonlimiting.

### EXAMPLES

### Example 1

### Production of Lipid-Rich Chlorella protothecoides using Two Methods of Supplying Glucose

The strain used is Chlorella protothecoides UTEX 250 (The Culture Collection of Algae at the University of Texas at Austin, USA).

Preculture:
500 ml of medium in a 21 Erlenmeyer flask;
composition of the medium (in g/l):

| | | |
|---|---|---|
| Macroelements (g/L) | Glucose | 40 |
| | K₂HPO₄ | 3 |
| | Na₂HPO₄ | 3 |
| | MgSO₄•7H₂O | 0.25 |
| | (NH₄)₂SO₄ | 1 |
| | Citric acid | 1 |
| | Clerol FBA 3107 (defoamer) | 0.1 |
| Microelements | CaCl₂•2H₂O | 30 |
| and vitamins (mg/l) | FeSO₄•7H₂O | 1 |
| | MnSO₄•1H₂O | 8 |
| | CoSO₄•7H₂O | 0.1 |
| | CuSO₄•5H₂O | 0.2 |
| | ZnSO₄•7H₂O | 0.5 |
| | H₃BO₃ | 0.1 |
| | Na₂MoO₄•2H₂O | 0.4 |
| | Thiamine•HCl | 1 |
| | Biotin | 0.015 |
| | B12 | 0.01 |
| | Calcium pantothenate | 0.03 |
| | p-Aminobenzoic acid | 0.06 |

Incubation takes places under the following conditions: duration: 72 h; temperature: 28° C.; stirring: 110 rpm (Infors Multitron incubator).

The preculture is subsequently transferred into a 30 l fermenter of Sartorius type.

Culture for Production of Biomass

The base medium is as follows:

| | | |
|---|---|---|
| Macroelements (g/l) | Glucose | 40 |
| | KH₂PO₄ | 0.9 |
| | NaH₂PO₄ | 0.7 |
| | MgSO₄•7H₂O | 1.7 |
| | (NH₄)₂SO₄ | 0.2 |
| | Clerol FBA 3107 (defoamer) | 0.3 |
| Microelements | CaCl₂•2H₂O | 20 |
| and vitamins (mg/l) | FeSO₄•7H₂O | 6 |
| | MnSO₄•1H₂O | 20 |
| | CoSO₄•7H₂O | 0.05 |
| | CuSO₄•5H₂O | 0.3 |
| | ZnSO₄•7H₂O | 25 |
| | H₃BO₃ | 7 |
| | Na₂MoO₄•2H₂O | 1 |
| | Inositol | 100 |
| | Choline chloride | 100 |
| | Thiamine•HCl | 3 |
| | Biotin | 0.05 |
| | B12 | 0.03 |
| | Calcium pantothenate | 0.1 |
| | p-Aminobenzoic acid | 0.1 |

The initial volume (Vi) of the fermenter is adjusted to 7 l after inoculation. It is brought to 15-20 l in the end.

The parameters for carrying out the fermentation are as follows:

| | |
|---|---|
| Temperature | 28° C. |
| pH | 6.8 (tests 1 and 2) or 5.2 (tests 3 and 4) with 28% w/w |
| | NH₃ and then 5N KOH |
| pO₂ | 30% (maintained with stirring) |
| Stirring | 300 rpm mini. |
| Air flow rate | 15 l/min |

Form of Supplying Glucose

Conventionally, when the residual concentration of glucose falls below 10 g/l, supplying with glucose is carried out so as to maintain the glucose content in the fermenter between 0 and 20 g/l.

This supplying is carried out starting from a concentrated 700 g/l glucose solution which is transferred into the fermenter using a peristaltic pump.

Supplying with glucose is managed according to two different methods:

1) Protocol 1: supplying glucose continuously with manually adjusted rate (tests 1 and 3)

In this type of feeding, the pump operates continuously.

As the evolution in the glucose requirements of the strain during the fermentation have been modeled, a model evolution profile of the flow rate of the pump is programmed (cf. FIG. 1, “% model pump” curve).

However, as the flow rates (of the order of 20 g/l/h at the maximum) and the cumulative amounts of glucose (approximately 1000 g/l) are very high, a slight discrepancy (less than the accuracy of the model) between the flow rate applied and the true rate of consumption of the strain rapidly results in a high variation in the residual glucose.

As is shown in FIG. 1, manual adjustments are thus frequently necessary (cf. FIG. 1, “% real pump” curve) during culturing in order to keep the residual glucose (cf. FIG. 1, “residual glucose” curve) within the desired range without being able to obtain perfect stability.

This method thus demands continuous control by an operator and the metabolism of the strain is sometimes limited by the availability of the glucose.

2) Protocol 2: supplying glucose by sequential and automatic additions as a function of the pO₂ (tests 2 and 4)

By the process in accordance with the invention, the additions of glucose are sequential and automated by virtue of an algorithm which controls the operation of the pump from the measurement of the content of dissolved oxygen (pO₂) in the fermentation medium using a dedicated probe.

The principle of the control is presented in FIG. 2.

When the concentration of glucose falls to 0 g/l in the fermenter, the oxygen consumption of the strain falls strongly, so that the pO₂ rapidly rises despite the fall in the stirring which is triggered by the algorithm for regulating the pO₂.

The detection of the rise in the pO₂ beyond a predetermined threshold (35%) triggers the startup of the glucose feed pump at its maximum speed for a predetermined time (6 minutes) in order to contribute an amount of glucose corresponding to a chosen concentration (10 g/l, corrected for the initial volume of the fermenter).

In this example, the period of time between the complete consumption of the glucose and the further addition is less than one minute.

The residual glucose is thus permanently maintained at a value greater than 0 and less than 10 g/l, without risk of limitation or of inhibition of the metabolism by the glucose.

In contrast to the manual protocol, the rate of the fermentation is at no point slowed down by the glucose.

Furthermore, this method is fully automated and operates without human monitoring being necessary.

Phase of Enriching in Lipids

In all cases, when 1000 g of glucose have been consumed and when the biomass has reached a concentration of 70 g/l, the aqueous ammonia is replaced with potassium hydroxide for the regulation of the pH. This makes it possible for the biomass to accumulate lipids.

Results:

| | | Residual | | | | |
|---|---|---|---|---|---|---|
| | | concen- | | | | |
| | | tration of | | | | |
| | Glucose | glucose | | Duration | Biomass | % |
| Test | feeding | (g/l) | pH | (h) | (g/l) | Lipids |
| 1 | Continuous | 0-30 | 6.8 | 96 | 177 | 45.4 |
| | addition with | | | | | |
| | manual | | | | | |
| | adjustments | | | | | |
| | (Protocol 1) | | | | | |
| 2 | Sequential | 0-10 | 6.8 | 86 | 175 | 45.3 |
| | and automatic | | | | | |
| | additions | | | | | |
| | (Protocol 2) | | | | | |
| 3 | Continuous | 0-30 | 5.2 | 94.5 | 182 | 44.6 |
| | addition with | | | | | |
| | manual | | | | | |
| | adjustments | | | | | |
| | (Protocol 1) | | | | | |
| 4 | Sequential | 0-10 | 5.2 | 88.8 | 176 | 44.5 |
| | and automatic | | | | | |
| | additions | | | | | |
| | (Protocol 2) | | | | | |

Protocol 2 according to the invention, that is to say supplying glucose by sequential and automatic additions, makes it possible to increase the productivity. This is because less fermentation time is necessary in order to achieve 70 g/l of biomass with the process according to the invention. This difference can be explained by the fact that the strain itself manages the supplying with glucose and that its metabolism is consequently never limited.

Furthermore, the differences in concentration of the residual glucose are lower with the process according to the invention.

### Example 2

### Production of Lipid-Rich Chlorella protothecoides with Pulsewise Addition of Glucose when the pO₂ Reaches 50% of Saturation

The culture medium and the operating conditions are identical to those of test 1 of example 1:

| | |
|---|---|
| Temperature | 28° C. |
| pH | 6.8 with 28% w/w NH₃ and then 5N KOH |
| pO₂ | 30% (maintained with stirring) |
| Stirring | 300 rpm mini. |
| Air flow rate | 15 l/min |

As in example 1, the additions of glucose are sequential and automated by virtue of an algorithm which controls the operation of the pump from the measurement of the dissolved oxygen content (pO₂) using a dedicated probe.

However, the pO₂ threshold which triggers the addition of glucose is in this instance higher: 50% of saturation instead of 35% of saturation.

That is to say, thus, a triggering threshold value 67% greater than the pressure of dissolved oxygen in the fermentation medium when the concentration of carbon-based source in the fermentation medium is not limiting.

The graph presented in FIG. 3 gives an example of implementation of the pulse technique. When the glucose concentration falls to a value of approximately 0 g/l in the fermenter, the oxygen consumption of the strain falls strongly, so that the pO₂ rapidly rises. Stirring is in this instance held at 400 rpm.

The detection of the rise in the pO₂ beyond 50% of saturation (i.e., 67% more than the value before the glucose becomes limiting) triggers the startup of the glucose feed pump at its maximum speed.

Final Results of this Test:
Duration: 90 h
Biomass: 176 g/l
Lipid content: 45.1%

Thus, despite the rise in the triggering threshold for the addition (50% of saturation, in comparison with 35% in example 1), the pulses are carried out very rapidly after the exhausting of the glucose and the culturing duration for achieving the desired concentration of biomass is not increased with respect to example 1.

### DESCRIPTION OF THE FIGURES

FIG. 1: Continuous feeding with glucose with manual adjustment

FIG. 2: Feeding with glucose by sequential and automatic additions—triggering threshold value: pO₂=35% (i.e., 16.7% greater than the value obtained when the concentration of carbon-based source in the fermentation medium is not limiting).

FIG. 3: Feeding with glucose by sequential and automatic additions—triggering threshold value: pO₂=50% (i.e., 67% greater than the value obtained when the concentration of carbon-based source in the fermentation medium is not limiting).

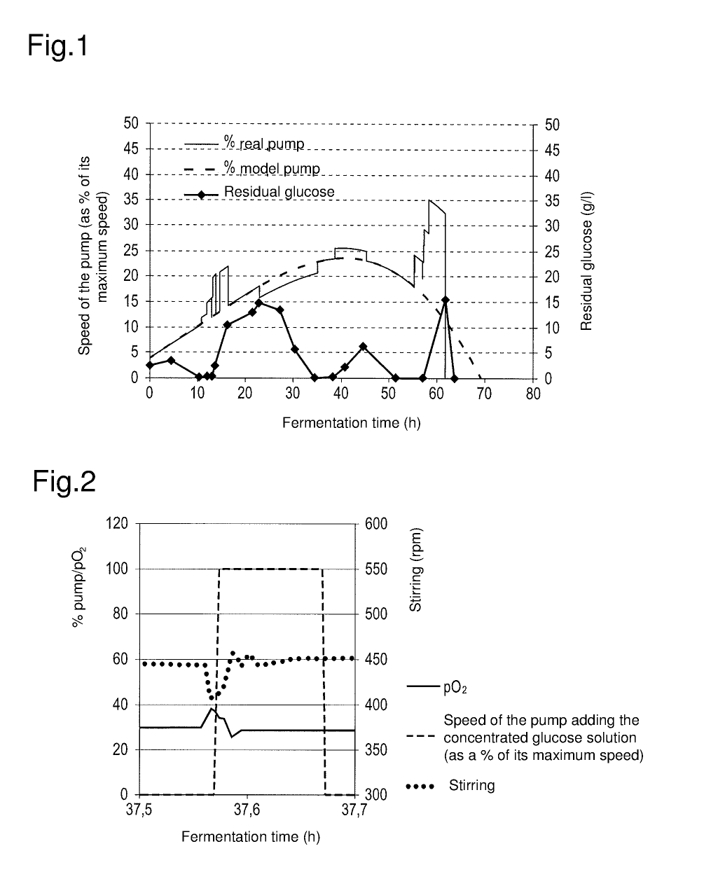

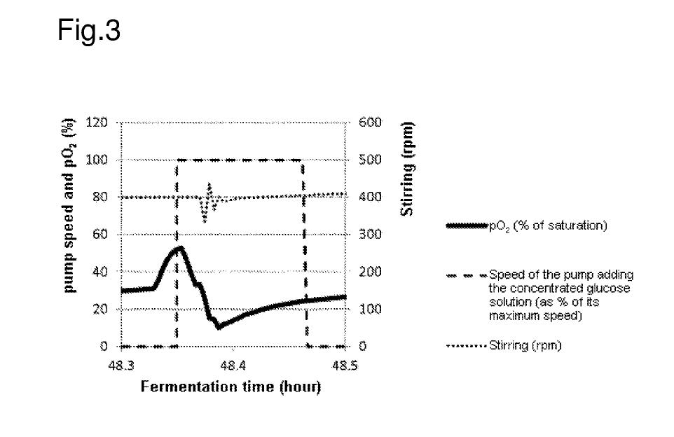

1.-13. (canceled

14. A process for the production of microalgae of the Chlorella genus by fed-batch fermentation, the method comprising:
providing a fermentation medium comprising a carbon-based substrate;
setting a threshold pO₂ value of the fermentation medium;
monitoring the pO₂ value of the fermentation medium to determine if the threshold pO₂ value is reached;
adding a carbon-based substrate to the fermentation medium when the pO₂ of the fermentation medium reaches or exceeds the threshold pO₂ value;
wherein the carbon-based substrate is added to the fermentation medium in less than 10 minutes; and
wherein when the pO₂ value of the fermentation medium is less than the threshold pO₂ value, no carbon-based substrate is added to the fermentation medium.

15. The process of claim 1, wherein the carbon-based substrate is added to the fermentation medium in less than 5 minutes or less than 1 minute.

16. The process of claim 1, wherein the threshold value is from 1 to 100% greater than the pO₂ value of the fermentation medium when the concentration of the carbon-based substrate is not limiting.

17. The process of claim 3, wherein the threshold value is 1 to 80%.

18. The process of claim 3, wherein the threshold value is 1 to 20%.

19. The process of claim 3, wherein the threshold value is 10 to 20%.

20. The process of claim 1, wherein the threshold pO₂ value is from 20% to 40% dissolved oxygen.

21. The process of claim 1, wherein the threshold pO₂ value is 30% dissolved oxygen.

22. The process of claim 1, wherein the microalga is selected from the group consisting of Chlorella protothecoides, Chlorella sorokiniana and Chlorella vulgaris.

23. The process of claim 9 wherein which the microalga is Chlorella protothecoides.

24. The process of claim 1, in which the carbon-based substrate is selected from the group consisting of glucose, acetate and ethanol.

24. The process of claim 11, wherein the carbon-based substrate is glucose.

26. The process of claims claim 1, wherein carbon-based substrate is maintained at a value of greater than 0 and less than 20 g/L.

27. A process for the production of Chlorella protothecoides by fed-batch fermentation, the method comprising:
Providing a fermentation medium comprising a carbon-based substrate;
setting a threshold pO₂ value of the fermentation medium;
monitoring the pO₂ value of the fermentation medium to determine if the threshold pO₂ value is reached;
adding a carbon-based substrate to the fermentation medium when the pO₂ of the fermentation medium reaches or exceeds the threshold pO₂ value;
wherein the carbon-based substrate is added to the fermentation medium in less than 10 minutes; and
wherein when the pO₂ value of the fermentation medium is less than the threshold pO₂ value, no carbon-based substrate is added to the fermentation medium.

28. The process of claim 27, wherein the threshold value is from 1 to 100% greater than the pO₂ value of the fermentation medium when the concentration of the carbon-based substrate is not limiting.

29. The process of claim 27, wherein the threshold pO₂ value is from 20% to 40% dissolved oxygen.

30. The process of claim 27, in which the carbon-based substrate is selected from the group consisting of glucose, acetate and ethanol.

31. The process of claim 30, wherein the carbon-based substrate is glucose.

32. A process for the production of Chlorella protothecoides by fed-batch fermentation, the method comprising:
Providing a fermentation medium comprising a carbon-based substrate;
setting a threshold pO₂ value of the fermentation medium of 20% to 40% dissolved oxygen;
monitoring the pO₂ value of the fermentation medium to determine if the threshold pO₂ value is reached;
adding a carbon-based substrate to the fermentation medium when the pO₂ of the fermentation medium reaches or exceeds the threshold pO₂ value;
wherein the carbon-based substrate is added to the fermentation medium in less than 10 minutes; and
wherein when the pO₂ value of the fermentation medium is less than the threshold pO₂ value, no carbon-based substrate is added to the fermentation medium.

33. The process of claim 32, wherein the carbon-based substrate is glucose.
